(19) United States
(12) Patent Application Publication
(10) Pub. No.: US 2015/0297488 A1
(43) Pub. Date: Oct. 22, 2015

(54) COMPOSITION COMPRISING A DICARBONYL COMPOUND AND METHOD FOR SMOOTHING THE HAIR USING THIS COMPOSITION

(71) Applicant: L'OREAL, Paris (FR)

(72) Inventors: Camila Biato, Rio de Janeiro (BR); Liliane Silvestre, Rio de Janeiro (BR)

(21) Appl. No.: 14/441,206

(22) PCT Filed: Nov. 7, 2013
(86) PCT No.: PCT/FR2013/052668

§ 371 Date: May 7, 2015

(30) Foreign Application Priority Data
Nov. 9, 2012 (FR) 1260658

(51) Int. Cl. A61K 8/44; A61K 8/365; A61K 8/73; A61Q 5/04

(52) U.S. Cl. CPC A61K 8/442; A61Q 5/04; A61K 8/365; A61K 8/731; A61K 2800/805

(57) ABSTRACT

The present invention relates to a composition comprising:
at least one dicarbonyl compound of formula (I) below and/or derivatives thereof and/or hydrates thereof and/or the corresponding salts:

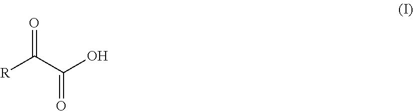

in which formula (I):
R represents an atom or group chosen from i) hydrogen, ii) carboxyl —C(O)OH, iii) optionally substituted linear or branched C₁-C₆ alkyl, iv) optionally substituted phenyl, v) optionally substituted benzyl, vi) an indolyl radical, and vii) an imidazolylmethyl radical, and tautomers thereof,
   at least one cellulose-based polymer and
   at least one amphoteric surfactant,
      the composition having a pH of less than or equal to 4.

The invention also relates to a process for straightening keratin fibers such as the hair, which comprises the application to the hair of the composition of the invention, followed by a straightening step using a straightening iron at a temperature of at least 150° C., preferably between 150 and 250° C.

The present invention relates to a cosmetic composition, especially a hair composition, based on particular dicarbonyl compounds, and also to a process for straightening keratin fibers such as the hair, using this composition.

In the hair field, consumers wish to have available compositions which make it possible to introduce a temporary change to their head of hair, while targeting good persistence of the effect produced. In general, it is desired for the change to withstand shampooing for at least fifteen days or even more, depending on the nature of said change.

Treatments already exist for modifying the color or shape of the hair and also, to a certain extent, the texture of the hair. One of the treatments known for modifying the texture of the hair consists in combining heat and a composition comprising formol. This treatment is especially effective for imparting a better appearance to damaged hair and/or for treating long hair and curly hair.

The action of formol is associated with its ability to crosslink proteins by reaction on the nucleophilic sites thereof. The heat used may be that of an iron (flat tongs or crimping iron), the temperature of which may generally be up to 200° C. or more. However, it is increasingly sought to avoid the use of such substances, which may prove to be aggressive to the hair and other keratin materials.

Patent application WO 2011/104 282 thus proposed a novel process for semi-permanently straightening the hair, which consists in applying an α-keto acid solution to the hair for 15 to 120 minutes, then drying and, finally, straightening the head of hair with an iron at a temperature of about 200° C. The α-keto acid employed is preferably glyoxylic acid.

However, it has been noted that the use of glyoxylic acid can give rise to significant limitations; in particular, when the scalp is sensitive and/or irritated. Its volatility, amplified by the use of heat (iron), can also be a problem.

It is already known practice to use glyoxylic acid esters in hair compositions, in particular in hair dye compositions, as described in document DE19859722, and in reducing compositions, as described in document DE19860239.

However, the efficacy of these compositions is insufficient.

The aim of the invention is to develop a straightening/relaxing composition which is stable over time and which makes it possible to straighten/relax and/or reduce the volume of keratin fibers in an efficient and persistent manner while limiting damage to the hair, while at the same time retaining comfort at the time of application for the user of the composition, but also for the hairdresser who applies it.

Thus, one subject of the present invention is a composition comprising
at least one dicarbonyl compound corresponding to formula (I) below and/or derivatives thereof and/or hydrates thereof and/or the corresponding salts:

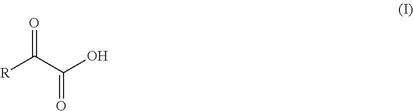

in which formula (I):
R represents an atom or group chosen from i) hydrogen, ii) carboxyl —C(O)—OH, iii) linear or branched C₁-C₆ alkyl which is optionally substituted, preferably with at least one hydroxyl —OH radical, carboxyl radical or halogen such as Br; iv) optionally substituted phenyl, v) optionally substituted benzyl, iv) and v) preferably being optionally substituted with at least one —OH or —C(O)—OH radical; vi) an indolyl radical, and vii) an imidazolylmethyl radical and tautomers thereof such as

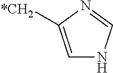

with * representing the part linked to the rest of the molecule,
at least one cellulose-based polymer and
at least one amphoteric surfactant,
   the composition having a pH of less than or equal to 4.

A subject of the invention is also a process for straightening keratin fibers, in particular the hair, which comprises the application to said fibers of the composition of the invention, followed by a straightening step using a straightening iron at a temperature of at least 150° C., preferably between 150 and 250° C.

The composition of the invention is stable. The composition of the invention, and the process for treating keratin fibers using the compounds of formula (I), the cellulose-based polymers and amphoteric surfactants as defined previously, allow good straightening of keratin fibers while limiting the degradation of these keratin fibers, even when the application of the composition(s) is followed by a heat treatment, especially by means of a hair-straightening iron, and have an appreciated working quality, especially without excessive vaporization of the composition at the time of straightening. The composition and the process for treating keratin fibers according to the invention also make it possible to limit the change in the color of the fibers and also the problems of breakage of said fibers such as the hair. The composition and the process of the invention will also improve the physical properties of the hair, by durably reducing the frizziness effect of the hair.

In that which follows, the expression “at least one” is equivalent to the expression “one or more”.

Preferably, the composition according to the invention does not comprise any coloring agent or any reducing agent.

According to the present invention, the term “coloring agents” means agents for coloring keratin fibers, such as direct dyes, pigments or oxidation dye precursors (bases and couplers). If they are present, their content does not exceed 0.001% by weight relative to the total weight of the composition. Specifically, at such a content, only the composition would be dyed, i.e. no dyeing effect would be observed on the keratin fibers.

It is recalled that oxidation dye precursors, oxidation bases and couplers are colorless or sparingly colored compounds, which, via a condensation reaction in the presence of an oxidizing agent, give a colored species. With regard to direct dyes, these compounds are colored and have a certain affinity for keratin fibers.

According to the present invention, the term “reducing agent” means an agent that is capable of reducing the disulfide bonds of the hair, such as compounds chosen from thiols, alkaline sulfites, hydrides and phosphines.

In the present invention, the dicarbonyl compounds of formula (I) or derivatives thereof may be in free form, but also in their hydrate forms or in the form of their salts, preferably in free form or in the form of hydrates.

As “derivatives” of the dicarbonyl compounds of formula (I), mention may be made of esters of the carboxy group(s), amides of the carboxy group(s), and (thio)acetals and hemi(thio)acetals of the carbonyl function(s) of the compounds of formula (I), in free form or optionally in the form of salts or of hydrates, preferably in free form or in the form of hydrates.

The esters and amides may be synthesized via conventional esterification or amidation processes, from the corresponding acids well known to those skilled in the art.

The esters are obtained, for example, from the acids of formula (I) and from a mono- or polyalcohol.

The term “mono- or polyalcohol” means an organic compound comprising one hydroxyl group (monoalcohol) or at least two hydroxyl groups (polyalcohol or polyol); said hydroxylated organic compound possibly being aliphatic, acyclic, linear or branched, or (hetero)cyclic, such as sugars (monosaccharides or polysaccharides) or sugar alcohols.

More particularly, the polyalcohol comprises from 2 to 100 hydroxyl groups, preferentially from 2 to 20 hydroxyl groups, even more preferentially from 2 to 10 hydroxyl groups, and better still 2 or 3 hydroxyl groups.

Preferably, the mono- or polyalcohol is chosen from methanol, ethanol, propanol, isopropanol, butanol, hexanol, ethylene glycol, glycerol, dihydroxyacetone, glucose, sorbitol and menthol.

The amides are obtained, for example, from the acid of formula (I) and from an organic mono- or polyamine.

The term “mono- or polyamine” means an organic compound comprising one amino group (monoamine) or at least two (and preferably from 2 to 100, better still from 2 to 20) amino groups; said organic compound possibly being aliphatic, acyclic, linear or branched or (hetero)cyclic.

The term “amino group” means a primary amine group —NH₂, or a secondary amino group >NH.

The mono- or polyamine is preferably aliphatic.

This amine is preferably chosen from methylamine, ethylamine, propylamine, isopropylamine, butylamine, hexylamine, monoethanolamine, monopropanolamine, propane-1,2,3-triamine and diaminoacetone.

The acetals and hemiacetals of the acids of formula (I) may be obtained, for example, from the reaction of alcohols with blocked forms of the acids, followed by hydrolysis. The alcohols may be the same as those mentioned for the esters. The acetals may also be cyclic acetals.

The salts may be salts derived from the interaction of the compounds of formula (I) with acids or bases, it being possible for the acids or bases to be of organic or mineral nature.

Preferably, the salts are salts derived from the interaction of the compounds of formula (I) with bases. Alkali metal or alkaline-earth metal salts, and in particular the sodium salts, will in particular be mentioned.

In the rest of the description, all these variants of dicarbonyl compounds of formula (I) will be referred to as “dicarbonyl compounds and/or derivatives thereof”.

According to a particular embodiment of the invention, the dicarbonyl compound(s) are of formula (I) with R representing i) a hydrogen atom or ii) a linear or branched C₁-C₆ alkyl group optionally substituted with a carboxyl group.

According to a more particular embodiment, the compounds of formula (I) are chosen from glyoxylic acid, pyruvic acid, a derivative thereof, a salt thereof and a hydrate thereof.

Preferentially, the dicarbonyl compound(s) of formula (I) of the invention are chosen from glyoxylic acid and derivatives thereof: glyoxylic acid and its hydrate form (HO)₂CH—C(O)—OH, for example, the glyoxylic acid as an aqueous 50% solution sold by the company Merck.

As glyoxylic acid derivatives, mention may be made of glyoxylic acid esters, glyoxylic acid amides, glyoxylic acid (thio)acetals and hemi(thio)acetals, glyoxylic acid ester (thio)acetals and hemi(thio)acetals.

The glyoxylic acid esters are obtained, for example, from glyoxylic acid and a mono- or polyalcohol, especially those mentioned previously.

Preferably, the mono- or polyalcohol is chosen from methanol, ethanol, propanol, isopropanol, butanol, hexanol, ethylene glycol, glycerol, dihydroxyacetone, glucose, sorbitol and menthol.

By way of esters, mention may in particular be made of methyl glyoxylate, ethyl glyoxylate, glyceryl glyoxylate, dihydroxyacetone glyoxylate, glyceryl diglyoxylate or triglyoxylate, sorbitol mono-, di- or triglyoxylate, glucose mono-, di- or triglyoxylate, menthyl glyoxylate, and acetals, hemiacetals and hydrates thereof.

The glyoxylic acid amides are, for example, obtained from glyoxylic acid and an organic mono- or polyamine, especially those described previously.

This amine is preferably chosen from methylamine, ethylamine, propylamine, isopropylamine, butylamine, hexylamine, monoethanolamine, monopropanolamine, propane-1,2,3-triamine and diaminoacetone.

Mention may be made in particular of glyoxylic acid N-β-hydroxyethylamide and glyoxylic acid N-γ-hydroxypropylamide and the acetals, hemiacetals and hydrates thereof.

The glyoxylic acid (thio)acetals and hemi(thio)acetals may be obtained, for example, from the reaction of alcohols, for the acetals or hemiacetals, or of thiols, for the thioacetals or hemithioacetals, with blocked forms of glyoxylic acid, followed by hydrolysis. The alcohols may be the same as those mentioned for the esters. The thiols may be equivalents (referred to as mono- or polythiols) to the mono- or polyalcohols mentioned above, except for the fact that the hydroxyl function(s) of said mono- or polyalcohols are replaced with one or more thiol functions SH of the mono- or polythiols. The acetals or thioacetals may also be cyclic (thio)acetals.

Mention may be made in particular of dimethoxyacetic acid, diethoxyacetic acid, 1,3-dioxane-2-carboxylic acid and 1,3-dioxolane-2-carboxylic acid.

According to a particularly preferred embodiment, the compound of formula (I) is glyoxylic acid in hydrate form.

Thus, the process of the present invention is neither carried out with a step of permanent reshaping at a basic pH, nor based on a reducing agent. The compositions according to the invention may be in any of the formulation forms conventionally used, and in particular in the form of an aqueous, alcoholic or aqueous-alcoholic, or oily solution or suspension; a solution or a dispersion of the lotion or serum type; an emulsion, in particular of liquid or semi-liquid consistency, of the O/W, W/O or multiple type; a suspension or emulsion of soft consistency of cream (O/W) or (W/O) type; an aqueous or anhydrous gel, or any other cosmetic form.

These compositions may be packaged in pump-action bottles or in aerosol containers, so as to apply the composition in vaporized (lacquer) form or in the form of a mousse. Such packaging forms are indicated, for example, when it is desired to obtain a spray or a mousse, for treating the hair. In these cases, the composition preferably comprises at least one propellant.

The compositions of the invention may be aqueous or anhydrous. They are preferably aqueous and then comprise water at a concentration ranging from 5% to 98%, better still from 5% to 50% and even better still from 10% to 40% by weight relative to the total weight of the composition.

According to one embodiment, the composition of the invention is in the form of an aqueous composition comprising from 0.1% to 20% of dicarbonyl compounds of formula (I) and/or derivatives thereof, preferably at least 3% of dicarbonyl compounds of formula (I) and/or derivatives thereof, preferentially from 3% to 10% by weight relative to the total weight of the composition.

According to the invention, the composition comprises at least one cellulose-based polymer. The cellulose-based polymers may be anionic, cationic, amphoteric or nonionic polymers.

According to the invention, the term “cellulose-based” polymer means any polysaccharide compound having in its structure sequences of glucose residues linked together via β-1,4 bonds; in addition to unsubstituted celluloses, the cellulose derivatives may be anionic, cationic, amphoteric or nonionic. Thus, the cellulose-based polymers of the invention may be chosen from unsubstituted celluloses, including those in a microcrystalline form, and cellulose ethers. Among these cellulose-based polymers, cellulose ethers, cellulose esters and cellulose ester ethers are distinguished. Among the cellulose esters are mineral esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/mineral esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

According to a first variant, the cellulose-based polymer(s) are nonionic. By way of example, mention may be made of (C₁-C₄)alkylcelluloses, such as methylcelluloses and ethylcelluloses (for example, Ethocel standard 100 Premium from Dow Chemical); (poly)hydroxy(C₁-C₄)alkylcelluloses, such as hydroxymethylcelluloses, hydroxyethylcelluloses (for example, Natrosol 250 HHR provided by Aqualon) and hydroxypropylcelluloses (for example, Klucel EF from Aqualon); mixed (poly)hydroxy(C₁-C₄)alkyl(C₁-C₄)alkylcelluloses, such as hydroxypropylmethylcelluloses (for example, Methocel E4M from Dow Chemical), hydroxyethylmethylcelluloses, hydroxyethylethylcelluloses (for example, Bermocoll E 481 FQ from Akzo Nobel) and hydroxybutylmethylcelluloses.

According to a second variant, the cellulose-based polymer(s) are anionic. Among the anionic cellulose ethers, mention may be made of (poly)carboxy(C₁-C₄)alkylcelluloses and salts thereof. Examples that may be mentioned include carboxymethylcelluloses, carboxymethylmethylcelluloses (for example Blanose 7M from the company Aqualon) and carboxymethylhydroxyethylcelluloses, and the sodium salts thereof.

According to a third variant, the cellulose-based polymer(s) are cationic. Among the cationic cellulose ethers, mention may be made of cationic cellulose derivatives such as cellulose copolymers or cellulose derivatives grafted with a water-soluble quaternary ammonium monomer, and described in particular in U.S. Pat. No. 4,131,576, such as (poly)hydroxy(C₁-C₄)alkyl celluloses, for instance hydroxymethyl-, hydroxyethyl- or hydroxypropylcelluloses grafted especially with a methacryloylethyltrimethylammonium, methacrylamidopropyltrimethylammonium or dimethyldiallylammonium salt. The commercial products corresponding to this definition are more particularly the products sold under the names Celquat® L 200 and Celquat® H 100 by the company National Starch.

According to a particular embodiment, the cellulose-based polymers of the invention are nonionic cellulose-based polymers comprising alkyl chains comprising from 1 to 6 carbon atoms. Preferably, the cellulose-based polymer(s) of the invention are chosen from cellulose ethers, in particular hydroxyalkylalkylcelluloses, such as (poly)hydroxy(C₁-C₄)alkyl (C₁-C₄)alkylcelluloses, in particular hydroxypropylmethylcelluloses (for example, Methocel F4M from Dow Chemical), hydroxyethylmethylcelluloses, hydroxyethylethylcelluloses (for example, Bermocoll E 481 FQ from Akzo Nobel) and hydroxybutylmethylcelluloses.

The cellulose-based polymers according to the present invention are cellulose-based polymers which preferably do not comprise a fatty chain, i.e. which preferably do not comprise a chain comprising more than 10 carbon atoms.

The cellulose-based polymer(s) of the invention may be present in the dye composition of the invention in contents ranging from 0.05% to 10% by weight, in particular from 0.1% to 5% by weight and better still from 0.1% to 3% by weight, relative to the total weight of the composition.

According to a particular embodiment, the composition comprises at least one amphoteric or zwitterionic surfactant.

In particular, the amphoteric or zwitterionic surfactant(s), which are preferably non-silicone, which may be used in the present invention may especially be derivatives of optionally quaternized aliphatic secondary or tertiary amines, in which derivatives the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines.

Among the optionally quaternized secondary or tertiary aliphatic amine derivatives that may be used, as defined above, mention may also be made of the compounds of respective structures (B1) and (B2) below:

Rₐ—C(O)—NH—CH₂—CH₂—N⁺(R_{b})(R_{c})—CH₂C(O)O⁻,M⁺,X⁻ (B1)
in which formula:
   Rₐ represents a C₁₀-C₃₀ alkyl or alkenyl group derived from an acid Rₐ—COOH preferably present in hydrolyzed copra oil, or a heptyl, nonyl or undecyl group;
   R_{b} represents a β-hydroxyethyl group; and
   R_{c} represents a carboxymethyl group;
   M⁺ represents a cationic counterion derived from an alkali metal or alkaline-earth metal, such as sodium, an ammonium ion or an ion derived from an organic amine, and
   X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylaryl sulfonates, in particular methyl sulfate and ethyl sulfate; or alternatively M⁺ and X⁻ are absent;

R_{a′}—C(O)—NH—CH₂—CH₂—N(B)(B′) (B2)
in which formula:
   B represents the group —CH₂—CH₂—O—X′;
   B′ represents the group —(CH₂)_{z}Y′, with z=1 or 2;
   X′ represents the group —CH₂—C(O)OH, —CH₂—C(O)OZ′, —CH₂—CH₂—C(O)OH or —CH₂—CH₂—C(O)OZ′, or a hydrogen atom;
   Y′ represents the group —C(O)OH, —C(O)OZ′, —CH₂—CH(OH)—SO₃H or the group —CH₂—CH(OH)—SO₃—Z′;
   Z′ represents a cationic counterion derived from an alkali metal or alkaline-earth metal, such as sodium, an ammonium ion or an ion derived from an organic amine;
   R_{a′} represents a C₁₀-C₃₀ alkyl or alkenyl group derived from an acid R_{a′}—C(O)OH, which is preferably present in coconut oil or in hydrolyzed linseed oil, an alkyl group, especially a C₁₇ group and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of the compounds of formula (B′2):

R_{a″}—NH—CH(Y″)—(CH₂)n-C(O)—NH—(CH₂)n′-N(R_{d})(Rₑ) (B′2)
in which formula:
   Y″ represents the group —C(O)OH, —C(O)OZ″, —CH₂—CH(OH)—SO₃H or the group —CH₂—CH(OH)—SO₃—Z″;
   R_{d} and Rₑ represent, independently of each other, a C₁-C₄ alkyl or hydroxyalkyl radical;
   Z″ represents a cationic counterion derived from an alkali metal or alkaline-earth metal, such as sodium, an ammonium ion or an ion derived from an organic amine;
   R_{a″} represents a C₁₀-C₃₀ alkyl or alkenyl group derived from an acid R_{a″}—C(O)OH, which is preferably present in coconut oil or in hydrolyzed linseed oil;
   n and n′, independently of each other, denote an integer ranging from 1 to 3.

Among the compounds of formula (B′2), mention may be made of the compound classified in the CTFA dictionary under the name sodium diethylaminopropyl cocoaspartamide and sold by the company Chimex under the name Chimexane HB.

Among the amphoteric or zwitterionic surfactants mentioned above, use is preferably made of the surfactants of formula (II) alone, or as mixtures.

In accordance with a particular embodiment of the invention, the content of amphoteric or zwitterionic surfactant(s) ranges from 0.05% to 30% by weight, preferably from 0.5% to 10% by weight and more preferably from 0.1% to 5% by weight, relative to the total weight of the composition.

The composition of the invention may also comprise at least one common cosmetic ingredient, chosen especially from propellants; oils; solid fatty substances and in particular C₈-C₄₀ esters; C₈-C₄₀ acids; C₈-C₄₀ alcohols; surfactants other than those described previously; sunscreens; moisturizers; antidandruff agents; antioxidants; chelating agents; nacreous agents and opacifiers; plasticizers or coalescers; fillers; silicones and in particular polydimethylsiloxanes; polymeric or non-polymeric thickeners; gelling agents; emulsifiers; polymers, in particular conditioning or hairstyling polymers; fragrances; basifying agents or acidifying agents; silanes; crosslinking agents. The composition can, of course, comprise several cosmetic ingredients appearing in the above list.

The composition may in particular comprise one or more organic solvents, in particular water-soluble solvents, such as C₁-C₇ alcohols; mention may in particular be made of C₁-C₇ aliphatic monoalcohols, C₆-C₇ aromatic monoalcohols, C₃-C₇ polyols or C₃-C₇ polyol ethers, which may be employed alone or as a mixture with water.

Depending on their nature and the purpose of the composition, the normal cosmetic ingredients can be present in normal amounts which can be easily determined by those skilled in the art and which can be, for each ingredient, between 0.01% and 80% by weight. Those skilled in the art will take care to choose the ingredients included in the composition, and also the amounts thereof, such that they do not harm the properties of the compositions of the present invention.

The pH of the composition is less than 4 and preferably ranges from 1 to 3, better still from 1.7 to 3.

The composition according to the invention is preferably in the form of styling or care gels, care lotions or creams, conditioners, masks or sera.

The composition according to the invention may be obtained by mixing several compositions.

The process of the invention comprises the application of the composition described previously, followed by a step of straightening the hair with an iron. Straightening with an iron is known from the prior art. It consists in straightening the hair with flat heating tongs, which are generally metallic. The straightening irons are generally used at a temperature ranging from 150 to 250° C.

The process of the invention may comprise other intermediate steps aimed at improving the straightening of the hair.

According to a particular embodiment, the process of the invention comprises the application of the composition of the invention to dry hair, and a time of contact of the composition with the hair of between 10 and 60 minutes and preferably between 20 and 40 minutes. After this leave-on time, straightening with a brush and with a hairdryer (blow-drying) is performed. The hair is then straightened with a straightening iron at a temperature of between 150 and 250° C. and preferably from 210 to 230° C.

The process of the invention may comprise the application of other hair agents as a pretreatment or post-treatment to the glyoxylic acid. In particular, it may comprise the application of a conditioning care product as a post-treatment.

According to another embodiment, the hair straightening process comprises a step of washing the hair and then of drying with a hairdryer before applying the composition of the invention. According to this particular embodiment, the steps described above are then encountered, such as the contact time of the composition, the blow-drying, the straightening with a straightening iron, the application of a conditioning agent and the rinsing, all these steps possibly being performed independently of each other. According to a particular embodiment, the straightening with a straightening iron is performed in several passes on the hair, in general 8 to 10 passes.

### EXAMPLE

Composition according to the invention: (the contents are indicated as crude starting material (in grams of the commercial product indicated))

| | |
|---|---|
| Hydroxypropylmethylcellulose (Methocel F4M from Dow | 1 |
| Chemical) | |
| Glyoxylic acid (50% aqueous solution) | 16 |
| Disodium cocoamphodiacetate (sodium N-cocoylamidoethyl-N- | 2 |
| ethoxycarboxymethyl glycinate) 30% aqueous solution (Miranol | |
| C2M Conc. NP from Rhodia) | |
| Crosslinked ethyltrimethylammonium methacrylate chloride | 2.5 |
| homopolymer, as a dispersion in a mix. of esters at 50%; | |
| (Salcare SC 96 from BASF) | |
| Lactic acid | 2.5 |
| Sodium hydroxide (10% aqueous solution) | 7 |
| Bisamino PEG/PPG-41-3 aminoethyl PG-propyl dimethicone at | 2 |
| 30% AM (Silsoft A 843 from Momentive Performance Materials) | |
| Water | qs |

*pH 2.2 ± 0.2

The above composition is applied to a lock of hair having a substantial level of curliness. After a leave-on time of 20 minutes on the hair, blow-drying is performed with a hairdryer and the lock is then straightened using a straightening iron. The evolution of fumes during the use of the iron is low.

The lock is then washed, and then dried with a hairdryer. A straight lock of hair free of curliness is thus obtained.

1.-14. (canceled)

15. A cosmetic composition comprising
at least one component chosen from dicarbonyl compounds of formula (I), derivatives thereof, hydrates thereof, or salts thereof: 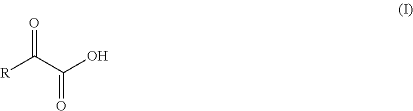
wherein in formula (I):
R represents an atom or group chosen from i) hydrogen, ii) carboxyl —C(O)OH, iii) linear or branched C₁-C₆ alkyl which is optionally substituted, iv) optionally substituted phenyl, v) optionally substituted benzyl, vi) an indolyl group, or vii) an imidazolylmethyl group and tautomers thereof;
at least one cellulose-based polymer; and
at least one surfactant chosen from amphoteric surfactants or zwitterionic surfactants,
wherein the cosmetic composition has a pH of less than or equal to about 4.

16. The cosmetic composition according to claim 15, wherein R is chosen from a hydrogen atom or a linear or branched C₁-C₆ alkyl group, optionally substituted with a carboxyl group.

17. The cosmetic composition according to claim 15, wherein the at least one component is chosen from glyoxylic acid and pyruvic acid, derivatives thereof, salts thereof, or hydrates thereof.

18. The cosmetic composition according to claim 15, wherein the at least one component is chosen from glyoxylic acid esters, glyoxylic acid amides, glyoxylic acid acetals, glyoxylic acid hemiacetals, glyoxylic acid thioacetals, glyoxylic acid hemithioacetals, glyoxylic acid ester acetals, glyoxylic acid ester hemiacetals, glyoxylic acid ester thioacetals, glyoxylic acid ester hemithioacetals, or derivatives thereof, hydrates thereof, or salts thereof.

19. The cosmetic composition according to claim 15, wherein the at least one component is glyoxylic acid in hydrate form.

20. The cosmetic composition according to claim 15, wherein the total amount of the component chosen from dicarbonyl compounds of formula (I), derivatives thereof, hydrates thereof, or salts thereof ranges from about 0.1% to about 20% by weight, relative to the total weight of the cosmetic composition.

21. The cosmetic composition according to claim 15, wherein the total amount of the component chosen from dicarbonyl compounds of formula (I), derivatives thereof, hydrates thereof, or the salts thereof ranges from about 3% to about 10% by weight, relative to the total weight of the cosmetic composition.

22. The cosmetic composition according to claim 15, wherein the at least one cellulose-based polymer is nonionic.

23. The cosmetic composition according to claim 15, wherein the at least one cellulose-based polymer is chosen from (C₁-C₄)alkylcelluloses, polyhydroxy(C₁-C₄)alkylcelluloses, hydroxy(C₁-C₄)alkylcelluloses, mixed hydroxy(C₁-C₄)alkyl-(C₁-C₄)alkylcelluloses, or mixed polyhydroxy(C₁-C₄)alkyl-(C₁-C₄)alkylcelluloses.

24. The cosmetic composition according to claim 15, comprising at least one amphoteric surfactant, wherein the amphoteric surfactant is chosen from compounds of formula (B1):
Rₐ—C(O)—NH—CH₂—CH₂—N⁺(R_{b})(R_{c})—CH₂C(O)O⁻,M⁺,X⁻ (B1)
wherein in formula (B1):
Rₐ is chosen from a C₁₀-C₃₀ alkyl or alkenyl group derived from an acid Rₐ—COOH, a heptyl group, a nonyl group, or an undecyl group;
R_{b} represents a β-hydroxyethyl group;
R_{c} represents a carboxymethyl group;
M⁺ is chosen from a cationic counterion derived from an alkali metal or an alkaline-earth metal, a sodium ion, an ammonium ion, or an ion derived from an organic amine; and
X⁻ is chosen from an organic or mineral anionic counterion, halides, acetates, phosphates, nitrates, (C₁-C₄)alkyl sulfates, (C₁-C₄)alkyl- or (C₁-C₄)alkylaryl sulfonates, methyl sulfate, or ethyl sulfate;
wherein M⁺ and X⁻ are optionally absent.

25. The cosmetic composition according to claim 15, comprising at least one amphoteric surfactant, wherein the amphoteric surfactant is chosen from compounds of formula (B2) below:
R_{a′}—C(O)—NH—CH₂—CH₂—N(B)(B′) (B2)
wherein in formula (B2):
B represents the group —CH₂—CH₂—O—X′;
B′ represents the group —(CH₂)_{z}Y′, with z=1 or 2;
X′ is chosen from —CH₂—C(O)OH, —CH₂—C(O)OZ′, —CH₂—CH₂—C(O)OH, —CH₂—CH₂—C(O)OZ′, or a hydrogen atom;
Y′ is chosen from —C(O)OH, —C(O)OZ′, —CH₂—CH(OH)—SO₃H, and —CH₂—CH(OH)—SO₃—Z′;
Z′ is chosen from a cationic counterion derived from an alkali metal or an alkaline-earth metal, a sodium ion, an ammonium ion, or an ion derived from an organic amine; and
R_{a′} is chosen from a C₁₀-C₃₀ alkyl or alkenyl group derived from an acid R_{a′}—C(O)OH, an alkyl group, a C₁₇ group, an iso C₁₇ group, or an unsaturated C₁₇ group.

26. The cosmetic composition according to claim 25, wherein R_{a′} represents a C₁₀-C₃₀ alkyl or alkenyl group derived from an acid R_{a′}—C(O)OH, which is present in coconut oil or in hydrolyzed linseed oil.

27. The cosmetic composition according to claim 15, comprising at least one amphoteric surfactant, wherein the amphoteric surfactant is chosen from compounds of formula (B′2) below:
R_{a″}—NH—CH(Y″)—(CH₂)n-C(O)—NH—(CH₂)n′-N(R_{d})(Rₑ) (B′2)
wherein in formula (B′2):
Y″ is chosen from —C(O)OH, —C(O)OZ″, —CH₂—CH(OH)—SO₃H, or —CH₂—CH(OH)—SO₃—Z″;
R_{d} and Rₑ, independently of each other, are each chosen from a C₁-C₄ alkyl or hydroxyalkyl group;
Z″ is chosen from a cationic counterion derived from an alkali metal or an alkaline-earth metal, a sodium ion, an ammonium ion, or an ion derived from an organic amine;
R_{a″} represents a C₁₀-C₃₀ alkyl or alkenyl group derived from an acid R_{a″}—C(O)OH; and
n and n′ denote, independently of each other, an integer ranging from 1 to 3.

28. The cosmetic composition according to claim 27, wherein R_{a″} represents a C₁₀-C₃₀ alkyl or alkenyl group derived from an acid R_{a″}—C(O)OH, which is present in coconut oil or in hydrolyzed linseed oil.

29. The cosmetic composition according to claim 15, wherein the total amount of surfactant chosen from amphoteric surfactant and/or zwitterionic surfactant ranges from about 0.05% to about 30% by weight, relative to the total weight of the cosmetic composition.

30. The cosmetic composition according to claim 15, wherein the total amount of surfactant chosen from amphoteric surfactant and/or zwitterionic surfactant ranges from about 0.1% to about 5% by weight, relative to the total weight of the cosmetic composition.

31. The cosmetic composition according to claim 15, wherein the cosmetic composition is aqueous.

32. The cosmetic composition according to claim 15, wherein the cosmetic composition is aqueous and comprises water in an amount ranging from about 5% to about 98% by weight, relative to the total weight of the cosmetic composition.

33. The cosmetic composition according to claim 15, wherein the cosmetic composition is aqueous and comprises water in an amount ranging from about 10% to about 40% by weight, relative to the total weight of the cosmetic composition.

34. A method for straightening keratin fibers, the method comprising:
applying to the hair a cosmetic composition comprising:
at least one component chosen from dicarbonyl compounds of formula (I), derivatives thereof, hydrates thereof, or salts thereof: 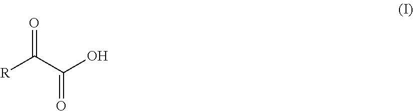
wherein in formula (I):
R represents an atom or group chosen from i) hydrogen, ii) carboxyl —C(O)OH, iii) linear or branched C₁-C₆ alkyl which is optionally substituted, iv) optionally substituted phenyl, v) optionally substituted benzyl, vi) an indolyl group, or vii) an imidazolylmethyl group and tautomers thereof;
at least one cellulose-based polymer; and
at least one surfactant chosen from amphoteric surfactants or zwitterionic surfactants,
wherein the cosmetic composition has a pH of less than or equal to 4;
allowing the cosmetic composition to contact the keratin fibers for a contact time ranging from about 10 and about 60 minutes; and
performing a straightening step using a straightening iron at a temperature of at least about 150° C.
